(19) United States
(12) Patent Application Publication
(10) Pub. No.: US 2022/0162541 A1
(43) Pub. Date: May 26, 2022

(54) ILLUMINATION SYSTEM FOR A PHOTOSYNTHETIC ORGANISM-GROWING MEDIUM

(71) Applicant: IXSCIENT LIMITED, Twickenham Middlesex (GB)

(72) Inventors: Nicholas Walker, Twickenham (GB); Michael Jackson, Twickenham (GB)

(21) Appl. No.: 17/594,058

(22) PCT Filed: Mar. 30, 2020
(86) PCT No.: PCT/GB2020/050847

§ 371 Date: Sep. 30, 2021

(30) Foreign Application Priority Data
Apr. 1, 2019 (GB) 1904561.6
Mar. 2, 2020 (GB) 2002996.3

(51) Int. Cl. C12M 1/00; C12M 1/34; G02B 6/036

(52) U.S. Cl. CPC C12M 41/06; C12M 21/02; G02B 2006/1218; C12M 41/12; G02B 6/03694; C12M 31/08

(57) ABSTRACT

In this invention there is provided an illumination system for a photosynthetic organism-growing medium. This system comprises a waterproof housing arranged to be inserted into a photosynthetic organism-growing medium and having a length along a longitudinal axis that is greater than a width. It also comprises a light source arranged to provide illumination along the length of the housing, and a diffuser arranged within the waterproof housing, the diffuser having a narrow end directed towards the light source and a wider end away from the light source and having a diffusive reflective surface arranged to diffusively reflect light from the light source to outside the housing.

### FIELD OF THE INVENTION

This invention relates to illumination systems for photosynthetic organisms such as algae, in particular to growing such organisms in a water based medium.

### BACKGROUND

Algae are a fast growing resource which only requires CO₂, nutrients and light to grow. Algae is a long-term sustainable source of biomass and oil which can be used for food, fuel and other bio products as well as for such uses as waste water processing.

Due to the commercial interest in culturing algae, it is beneficial to optimise the growth of algae. Well known methods and apparatus have made it possible to use artificial light to increase the yield of algae, when sunlight cannot be relied upon to provide the light needed. This allows algae to be grown in different environments, such as in water processing plants which operate 24 hours or in regions with low sunlight, where natural light is limited. Illumination systems which provide a uniform distribution of light are preferred to maximise growth rates. It is also necessary to optimise the light intensity, as under too intense light the algae may be photo-inhibited or die, and under too low intensity the algae growth is reduced.

Present methods of optimising algae growth include the use of LED solid state lighting to produce artificial light to the algae. This lighting is efficient and produces lower levels of heat than previous generations of lighting. One known design for the illumination of algae within a medium is an array of transparent vertical tubes spaced evenly throughout the medium extending from top to bottom. Each tube contains multiple evenly spaced light sources, for example strips of LEDs on a central heat conductor, such that light is emitted uniformly throughout the medium. This ensures that the algae medium is substantially illuminated throughout, rather than solely the topmost algae being illuminated and the light not reaching the depths of the medium.

We have appreciated that light sources produce heat, and so their use within the medium may result in the algae being heated above their threshold and being baked onto the surface of the tube. This baked algae is hard to remove and results in a decrease in outputted light intensity, and decreases uniformity due to the patches of baked algae positioned respective to the placement of the light source. The use of this apparatus therefore does not guarantee a uniform spread of light, and has a reduced efficiency after continued use.

In illumination systems the majority of problems encountered are related to the faults in the electrics, for example the light source. We have appreciated that the commonly used apparatuses in which the entire illumination system is placed within the medium creates difficulties. In order to fix the illumination system the entire system must be removed from the medium, which is difficult due to the need to have a substantially heavy system for it to be submerged.

To power the submerged illumination system, electrical power must also be conducted down into the fluid medium. As the lights are within the tube, this creates a safety issue if the tube leaks or breaks and the electrics are submerged in a fluid. Therefore, to increase the safety of the system the voltage is limited to a low value, such as less than 15 volts. This results in a limited power available, therefore reducing the number of light sources which can be used, or the intensity of each source. There is also a further power loss due to resistance in the wires which can reduce efficiency of the system, especially if the wires to the tubes or the tubes themselves are long.

In order to minimise some of these issues, other apparatuses used to optimise the growth of algae include the use of waveguides, through which light is transferred from a remote light source into the liquid medium. Such waveguides are generally solid and consisting of shaped blocks of a clear plastic such as acrylic. Examples of waveguides used are wedges and tapering rods. In these designs the waveguide itself is immersed in the medium, reducing the difficulty of fixing the light source, and eliminating the power loss through wiring. However, heat is still produced at the immersed waveguide, again causing algae to be baked onto the surface. Therefore, as in the other apparatus, intensity, uniformity and efficiency is again reduced.

### SUMMARY OF THE INVENTION

We have appreciated the need to improve the efficiency of the artificial light source used to grow photosynthetic organisms. In particular, we have appreciated the need for uniformity in the light outputted from the system, whilst controlling the heat output from the light source in order to maximise the photosynthetic organism production. Further, we have appreciated the need for such an apparatus whose use is possible below the surface of the photosynthetic organism-growing medium without the loss of uniformity of light. The need for a more efficient illumination system has also been appreciated and the present invention is able to be used with multiple types of light source, allowing the user to select the preferred light source chosen such that it is more efficient, or such that it is high powered and able to reach larger depths of the medium.

The invention is defined in the independent claims to which reference is directed. Some embodiments are defined in the dependent claims.

In particular, there is provided an illumination system for a photosynthetic organism-growing medium. This system comprises a waterproof housing arranged to be inserted into a photosynthetic organism-growing medium and having a length along a longitudinal axis that is greater than a width. It also comprises a light source arranged to provide illumination along the length of the housing, and a diffuser arranged within the waterproof housing, the diffuser having a narrow end directed towards the light source and a wider end away from the light source and having a diffusive reflective surface arranged to diffusively reflect light from the light source to outside the housing.

In operation of an embodiment of the invention light passes from the light source into the housing, in which the light is diffused by the diffuser. The diffuser spreads the light so that the entirety of the medium along the length of the housing is illuminated, rather than the majority of light being emitted from just one position. The housing has a length along a longitudinal axis which is greater than its width so that the housing extends down into the medium and increases the depth of photosynthetic organisms which are able to be illuminated. A benefit of this arrangement is that the light source and housing may be separate units allowing the lighting element to be outside the medium. In this way electrical components, for example the light source and heat management systems, may be outside of the medium, reducing safety risks and making it easier to fix problems with the electric elements as the entire system does not need to be removed from the medium. The use of a remote light source has the advantage that it can be removed, replaced or repaired without having to remove the components which are mounted within the photosynthetic organism growth volume.

In other arrangements of the invention the light source, and any additional features such as optical elements and/or heat management systems, are placed within the housing, also resulting in reducing safety risks by creating a water tight system. This arrangement has the advantage that the efficiency is increased by minimising the space between the light source and diffuser.

The housing is watertight so that the medium within the housing is constant, allowing the apparatus to be optimised for the specific light source and medium within the housing used.

Embodiments of the invention provide various advantages over other photosynthetic organism illumination systems. In these embodiments the system can comprise additional features which result in improvements in the growth rate of photosynthetic organisms, or usability of the system.

Optionally, the diffuser has a monotonically increasing width from near the light source to away from the light source. The advantage of the increase in width of the diffuser further down the housing is that it increases the probability of the light hitting the diffuser which counteracts the decrease in probability of the light reaching that depth. Therefore, the intensity of the light being scattered out of the housing is uniform along the length of the diffuser and thus ensures the necessary uniformity of light emission.

Optionally the diffuser is cone shaped. This has the effect that light is scattered uniformly along the length of the diffuser to outside the housing.

Optionally the diffuser has a circular base. This shape is simple to manufacture and has the effect that the light is scattered uniformly as there are no vertices on the diffuser. Optionally the diffuser is symmetrical about the longitudinal axis, having the effect that the spread of light from the diffuser is uniform in all directions around the diffuser.

Optionally the diffuser has a point at the end of the housing towards the light source, allowing scattering of light from the start of the housing.

Optionally the diffuser has a base at the end away from the light source, such that scattering occurs until the end of the housing.

Optionally the diffusive reflective surface comprises a diffusively reflective paint. This has the advantage that the surface may be evenly coated in a simple manner. Thus, the diffusion of light is more uniform.

Optionally the paint used contains a high concentration of barium sulphate. Barium sulphate has a high reflectance, up to 99%, and its white colour enables it to reflect all wavelengths, and is therefore compatible with all wavelengths of light.

Optionally the housing is completely transparent, having the effect that the light enters and leaves the housing without losing brightness and thus improving the efficiency.

Optionally the housing can be a plastic, such as acrylic, polycarbonate or PET or glass. This has the advantage that the materials are strong and able to withstand the pressures of being submerged in water.

Optionally the housing has a circular cross section. This has the advantage that the light is more evenly spread as there are no vertices on the housing and the housing is simple to manufacture.

Optionally the house comprises a light redirecting film. This improves the spread of light from the housing.

Optionally the light redirecting film is located around the inside perimeter of the housing. This has the advantage that it is not subject to the medium and is therefore more protected from being damaged.

Optionally the light directing film is a prismatic film with specified prism angles. This has the effect that the light redirection is optimised by reflecting light at certain angles but refracting light at others and the prism angles can be selected dependent on the required angles of light entering the medium.

Optionally the internal angle at the apex of the prisms is 90 degrees and they are arranged such that the prism grooves are oriented along the system axis.

Optionally the prisms are arranged such that light travelling along the axis of the system is reflected back into the housing, and light travelling close to orthogonal to the axis passes through the prismatic film.

Optionally the prisms run along the length of the film in the axial direction of the housing, thus improving the uniformity of the spread of light.

Optionally the prisms face inwards to improve the spread of the light.

Optionally the prismatic film has a selected critical angle, preferably allowing light waves between −24 and 24 degrees to the orthogonal. This angle optimises the effect of the redirecting film, allowing the optimised amount of light to be refracted.

Optionally the system comprises a heat management system arranged to remove heat generated by the light source. This has the advantage that the light source does not heat the photosynthetic organism growing-medium above its threshold, therefore killing the photosynthetic organisms and decreasing the yield and efficiency of the system.

Optionally the heat management system can comprise of any of the following, or any combinations of the following: a heat sink, fan, liquid cooling system, heat pipe, heat fin or an air cooled heat exchanger. These systems have the advantage that they remove the heat from the light source and can be chosen dependent on the use of the heat removed.

Optionally the heat management system is a liquid cooled system with the advantage that the heat management system and light source may be compact enough to fit inside the housing. This allows the distance between the light source and diffuser to be minimised and the efficiency is therefore increased. There is also the advantage that the whole illumination system can be watertight, providing the advantage that it can be used in external environments without damage due to rain.

Optionally the space between the light source and diffuser comprises a reflective film to minimise light loss before diffusion.

Optionally, the light source may be arranged within the housing or outside the housing and arranged to provide illumination into the housing.

Optionally the light source is a laser or an LED. The advantage of this is that the light source can be chosen dependent on the use of the system and the result required.

Optionally the light source emits a mixture of red and blue light. Photosynthetic organism growth is due to photosynthesis and this is maximised by the use of red and blue light in the correct proportions.

Optionally the system comprises a phosphor layer, this has the effect that the phosphor is able to change the wavelength of light received to a different one emitted, and therefore a more efficient blue light can be used and the phosphor layer converts the optimal amount of blue light to red. This can be more efficient than using a mixture wavelengths of LED light sources.

Optimally the phosphor produces a desired light spectrum. This optimises the growth of photosynthetic organisms.

Optionally the desired spectrum is a mix of wavelengths of light, arranged to optimise the photosynthesis of the photosynthetic organisms.

The desired spectrum is preferably a spectrally relevant light source for the media being grown . This is an effective light for the growth of photosynthetic organisms.

Optionally, when the light source is an LED, the phosphor can be applied directly onto the light source. This has the effect that the light spectrum is altered, and the type or amount of phosphor is able to be able to varied or replaced without removing the housing.

Optionally the phosphor layer is a film on the housing, with the advantage that the type of phosphor does not have to be able to withstand the heat of the light source.

Optionally the phosphor layer film is located adjacent to the inside of the housing so that the light reaches it after any diffusion from a light redirecting film, if present. This has the advantage that the phosphor film does not disperse the light before it is dispersed by the prismatic film and the optimal diffusive spread is achieved.

Optionally the phosphor films consists of a substrate and a coated material. This has the advantage that the layer is more durable.

Optionally the material consists of a UV curable acrylate in which inorganic phosphor has been suspended.

Optionally the system comprises optics arranged to direct light from the light source. These have the advantage that any light source can be used and the optics selected to direct light appropriately from the source.

Optionally the optics create a substantially collimated light source. This is advantageous as the light must be collimated enough that the diffusion and reflectance can be optimised and the spread of light is optimised.

Optionally the optics produce light that is diverging. This has the effect that if the light source is too collimated, such as a laser, the angle of light can be increased.

Optionally the optics produce light that is collimated. This has the advantage that light sources which are not sufficiently collimated can be used in the system.

Optionally the collimating optics is a reflective parabolic mirror. Optionally the collimating optics is a lens. These are able to be chosen such that the required collimation can be achieved.

Optionally the system can comprise an additional housing on top of the light illuminated housing and the light source is a laser. This has the effect that the light illuminated housing is further down in the medium without the light having to pass through as much water but instead passes through air. The use of a laser results in the light being powered enough to pass through the additional housing whilst also being able to illuminate the housing below.

Optionally the additional housing can be air filled with no additional elements. This enables the light to pass through uninterrupted.

Optionally the additional housing can contain an internally reflecting film mounted within the housing. This has the advantage that the efficiency is increased as no light escapes the additional housing on its way through.

Optionally the housing comprises a diffuser and an additional light source located below the laser. This allows the light to be diffused from the additional housing as well as the below laser system.

Optionally the light source is a circular arrangement illuminating the edge of the additional housing so that the light passes through the gap. This allows light to pass through the gap to the below housing.

Optionally there is a diverging optical plate mounted between the two housings, having the effect that the laser can be diverged to optimise the result of the diffusion in the lower housing.

Optionally the diffuser in the additional housing does not taper to a point.

Optionally the light source passes through the top of the diffuser which are arranged such that the diameter of the top and the light source are the same. This has the advantage that the light from the laser can pass through the additional housing without being diffused.

Optionally the optical element is some distance down the additional housing, having the effect that the laser is diverged once passing through the additional housing and thus optimising the diffusion of the light.

Optionally the diffuser is made of thin plastic and filled with a dense material, having the result that the weight of the housing part can be increased to optimise buoyancy.

Optionally diffuser can contain a material incorporating iron.

Optionally the diffuser is completely made form a heavy metal, having the result that the weight of the housing part can be increased to optimise buoyancy.

Optionally the diffuser can be made from stainless steel.

Optionally the photosynthetic organisms can be algae.

### BRIEF DESCRIPTION OF THE DRAWINGS

Some ways in which the invention may be performed are described in more detail by way of example with reference to the accompanying drawings, in which:

FIG. 1a: is an illustration of the illumination system in a first embodiment showing one possible shape of a diffuser;

FIG. 1b: is an illustration of the illumination system in the first embodiment showing an alternative possible shape of a diffuser;

FIG. 1c: is an illustration of the illumination system in the first embodiment showing an alternative arrangement in which the light assembly is inside the housing;

FIG. 1d: is an illustration of the illumination system in the first embodiment showing an alternative arrangement in which the light assembly is inside the housing and separated from the diffuser by a separating portion;

FIG. 2: is an illustration of the illumination system in a second embodiment wherein a phosphor layer is located directly on the light source;

FIG. 3: is an illustration of the illumination system in a third embodiment wherein a phosphor layer is located on the inside of the housing;

FIG. 4: illustrates three possible variations of a fourth embodiment in which there is an additional housing;

FIG. 5a: shows an alternative arrangement of collimated light beams for use with the fourth embodiment;

FIG. 5b: shows an alternative arrangement of collimated light beams for use with the fourth embodiment;

FIG. 6a: shows a first view of a light assembly; and

FIG. 6b: shows a second view of a light assembly.

### DETAILED DESCRIPTION

The embodiment of the invention described provides an arrangement to allow the illumination of photosynthetic organisms within a photosynthetic organism growing-medium. The invention optimises growth by improving the uniformity of the spread of light within the medium, as well as improving the efficiency of growth per watt of electricity.

There are a number of features of embodiments used to optimise the illumination of photosynthetic organisms. Specific examples of embodiments are described below. The embodiments disclosed herein are described in relation to an algae-growing medium, however it will be appreciated that this is solely an example and the embodiments are suitable for any photosynthetic organism growing-medium.

The embodiments proposed here overcome the problems presented by enabling the light source to be set at one end of the tube. By the use of the apparatus in some embodiments disclosed herein, and using the right light sources, the medium is able to be illuminated several metres below the surface.

The invention may be embodied in a variety of ways, such as to allow the use of a system to illuminate algae in varying depths of water by use of embodiments with different light sources and additional housings. Other embodiments allow the use of a variety of light sources, by using optional optical elements.

The present disclosure describes various arrangements of an algae illumination device embodying the present invention. The described arrangements allow for an increase in efficiency, safety and optimisation of uniformity of illumination. Embodiments disclosed herein provide the benefit of several optional features whose inclusion will provide the benefit of overcoming the limitations described earlier.

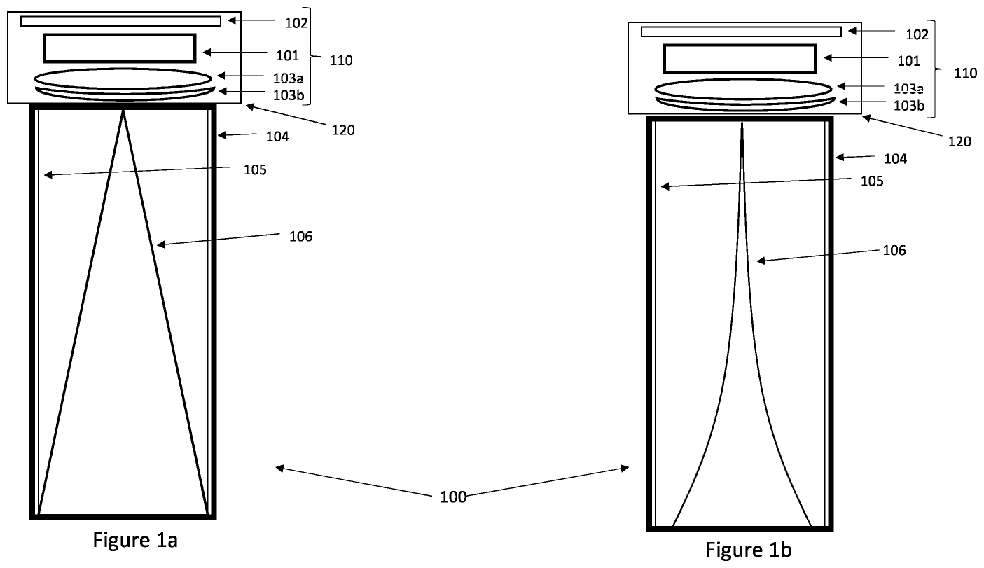

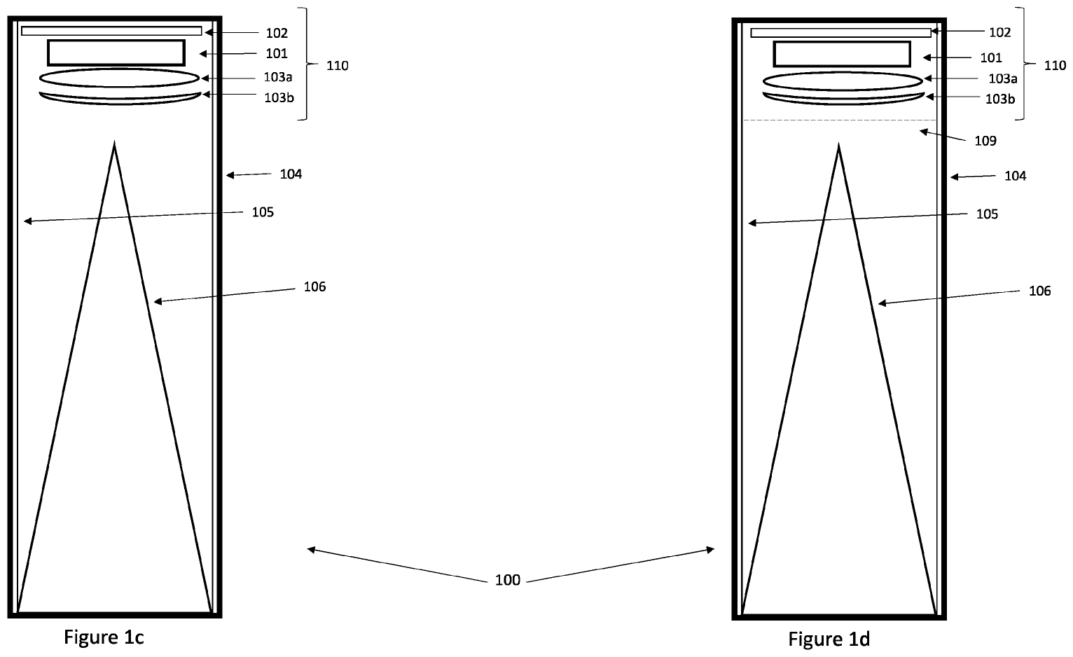

FIG. 1a shows an optical illumination system 100 for an algae growing medium illustrating one embodiment of the invention. FIG. 1b is an illustration of the same system as 1a, and is included to illustrate the alternative shapes of a diffuser of the system. As the two figures contain the same features, the following description applies to both figures. The system 100 may comprise a light source 101, a heat management system 102, an optical element 103a, here a collimating element, a further optical element 103b, here a diverging element, a diffuser 106, a waterproof housing 104, and a prismatic optical film 105. It will be appreciated that the system 100 according to the present invention may not necessarily include every module shown in FIG. 1, as will be described in the following paragraphs. It will be appreciated that such a system can be used within any medium for which a uniformed light is required.

In this first embodiment the illumination system 100 comprises a waterproof air-filled housing 104, arranged to be inserted into an algae growing medium. The use of an air-filled housing is optional, and it will be appreciated that the housing may be filled with any suitable medium. However, the use of air protects the features within the housing, as will be described later, from damage by water or another medium. The housing 104 is cylindrical in shape, and orientated such that it extends downwards, and the circular cross section is perpendicular to the longitudinal axis. The housing 104 is sufficiently transparent, such that light is able to enter and leave the housing 104. The housing 104 is able to be made of any material, provided that it is water tight, and is able to withstand the pressure at the depths that it will be placed at. Examples of such materials are a plastic, such as polycarbonate, acrylic or PET or glass. It is appreciated that the housing 104 is able to be up to several metres long, and in some cases is able to be submerged several metres below the surface of the algae growing medium.

The system 100 also comprises a light source 101 arranged such that it is situated at one end of the housing, either inside or outside of the housing 104, as shown in FIGS. 1a-1d. Preferably it is located outside of the medium to reduce any safety issues. The light source 101 and housing 104 are aligned such that the light shines into the housing 104, along the longitudinal axis, thus providing light along the length of the housing 104. As illustrated, the light source 101 may be contained within its own housing, forming a light assembly 110, and this may contain other features such as an optical system 103a-b and/or a heat management system 102. This results in an easy removal and replacement of features.

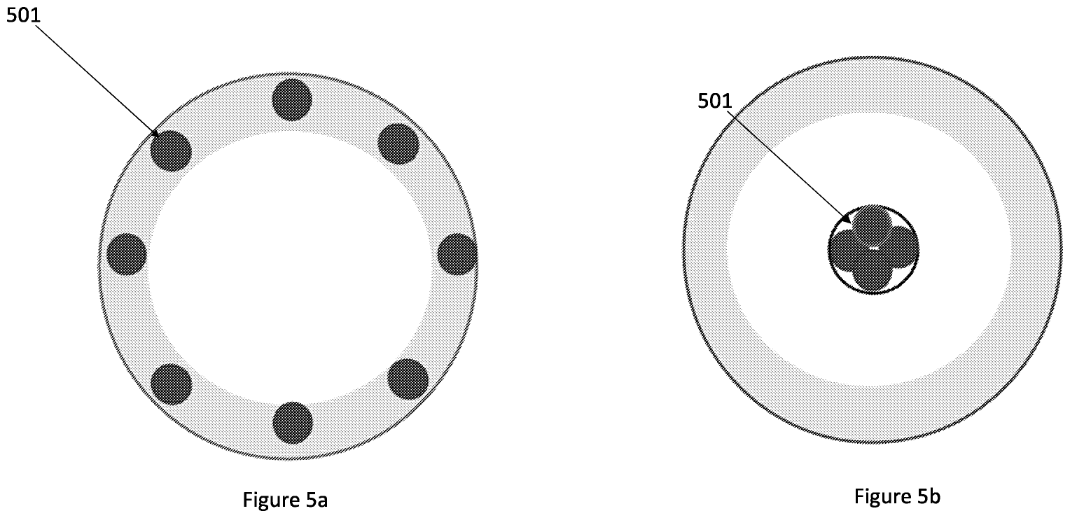

The light source 101 may be either an LED ora laser, dependent on the application for which the apparatus is being used. Algae grows by photosynthesis, for which light in the red (approx. 670 nm) and blue (approx. 450 nm) ranges are most effective. Therefore, it has been appreciated that to maximise growth per watt used by a light source 101, it is preferable to use a source which emits only the right spectrum. In order to achieve this there are multiple options, one of which is described in this embodiment, and the others are described in the later embodiments. One such option is that the light source 101 is an LED or laser diode mix producing a mixture of 420 nm-480 nm blue and 650 nm-690 nm red. This mixture is chosen to optimise the growth of algae within the medium, and as can be appreciated, this mixture is able to be varied dependent on the spectrum required. One example of a mix is a ratio of 4 red LEDs to one blue LED. This apparatus is not limited to a specific arrangement of the light source 101, but examples of a light source 101 used are LEDs arranged in a circular array and spaced evenly apart, such an example is known as a ‘cob’ in which individual pieces of LED are arranged in a circular formation to provide a high intensity light source. Alternatively, the LEDs or lasers can be arranged in a circle such that there is an area in the middle which does not provide illumination, as illustrated in FIG. 5a.

We have appreciated the need for a uniform distribution of light to maximise the growth of algae in a medium. Therefore, in the preferred embodiment the housing comprises a prismatic optical film 105 which redirects light as it leaves the housing 104. The prismatic film 105 comprises many solid prisms extending the length of the film which itself extends the length of the housing 104, located along the inside perimeter of the housing 104. The points of the prisms preferrably have an angle of 90 degrees with the result that the light behaves in the optimal way. The prisms preferentially face inwards. When the light refracts off the diffuser, it can be travelling in a range of 180 degrees, the use of a prismatic film 105 improves the spread of the angles when the light leaves the housing. The prisms on the film 105 have specified angles which allow a specific range of angles of incoming light to pass through the film. The film 105 acts as a mirror below its critical angle, and lets light through at an angle above its critical angle. This allows an optimal amount of the light deflected towards the housing 104 to be allowed out, whilst also ensuring that the light is uniformly spread along the length of the housing 104. For example, for a prismatic film 105 which allows light from a range of −24 to 24 degrees from the orthogonal of the axis of the system to leave the housing 104, light waves which are reaching the prism at 25 degrees, and therefore originated from further away from the point, will be deflected back into the housing 104 to be deflected off the diffuser 106 again and redistributed. This has the result that light travelling along the axis of the system is reflected back to the diffuser 106, and light that is travelling close to orthogonal to the axis of the system passes through. This improves the spread of the light as the light is pushed down the housing 104 and along with the use of a cone shape diffuser the length of the tube can be greater than in a system without such a film 105. It will be appreciated that the use of this film 105 is optional and with a highly collimated light source 101 and a short housing it would not be necessary. The prismatic film is not wavelength sensitive, and the critical angle varies a neglible amount between red and blue light, allowing the same film to be used for the whole light spectrum.

The system 100 also comprises a diffuser 106 arranged within the waterproof housing 104. The diffuser has a narrow end directed towards the light source 101 and a wider end away from the light source 101. The diffuser 106 also has a diffusive reflective surface arranged to diffusively reflect light from the light source 101 to outside of the housing 104. In the embodiment in FIG. 1a, the diffuser 106 is monotonic and cone shaped with a circular cross section. Its pointed end is at the light source 101 facing end of the housing 104 and its base is at the opposite end of the housing 104. The use of a monotonic, cone shaped diffuser has the advantage that the increasing size of the diameter improves the probability of light hitting the diffuser the further down it goes. This makes up for the lower probability of the light reaching further down the tube as the intensity decreases, and thus increases the uniformity of the spread of light down the entire length of the tube. It can be appreciated that the diffuser 106 shape and size can be varied to optimise the result with a chosen light source 101 and does neither have to be conical nor symmetrical, as can be seen in FIG. 1b and FIG. 5b. As illustrated in FIG. 1a the diffuser 106 can perfectly fit in the housing 104 and has no gap between its base and the axial side of the bottom of the housing 104. However, it can be appreciated that the diffuser can be chosen such that it does not fully fill the bottom of the housing, and in this case there may be an additional diffuser fitted around the base of the diffuser. The diffuser is not wavelength sensitive, allowing it to be used with the whole light spectrum.

We have appreciated that in some mediums there may be a difficulty in achieving the ideal weight of the system, such that it remains submerged within the medium, but is not too weighted that it would be difficult to remove if needed. Therefore, in some embodiments we have appreciated that additional weight would improve the use of the system. Therefore, the system can be optionally optimised by use of a weighted diffuser 106 within the housing 104. Examples of weighting include, but are not limited to, fabricating the cone out of a heavy metal such as stainless steel, or the diffuser 106 may be made of a thin plastic surface and be filled with concrete with additional fillers such as iron to increase its density.

The central diffuser 106 is coated with a diffusing surface with a very high reflectance, for example a white paint containing a high concentration of barium sulphate which is up to 99% reflective. Such types of paint are used to coat the interior of optical integrating spheres. White paint reflects all wavelengths of light and therefore any light source 101 is able to be used with the diffuser. Other surfaces can be used, such as a patterned aluminium, however the use of paint allows the thickness to be varied depending on the result required. Furthermore, unlike a surface such as aluminium, paint has no seam and therefore the light is more uniformly diffused. The use of a diffusive surface rather than a solely reflective surface results in not just creating one beam, but creates a spread of light in all directions, increasing uniformity and ensuring all algae is illuminated in the medium along the length of the cylinder.

The above arrangement describes the light assembly 110 located outside of the housing 104 in its own separate housing 120. In an alternative arrangement the light assembly 110 may be located inside the housing 104, at one end, as illustrated in FIGS. 1c and 1d. It will be appreciated that this is possible in any of the embodiment described herein. This arrangement has the advantage that construction of the system is simpler. In the case that the light assembly 110 is located within the housing 104, there may be another arrangement in which a separating portion 109 is located between the light assembly 110, such as in FIG. 1d, to prevent the ingress of dust when the light assembly is removed, and therefore does not need to be robust enough to be water tight. The separating portion 109 is preferably a completely transparent material through which the light from the source is able to pass without decreasing its efficiency.

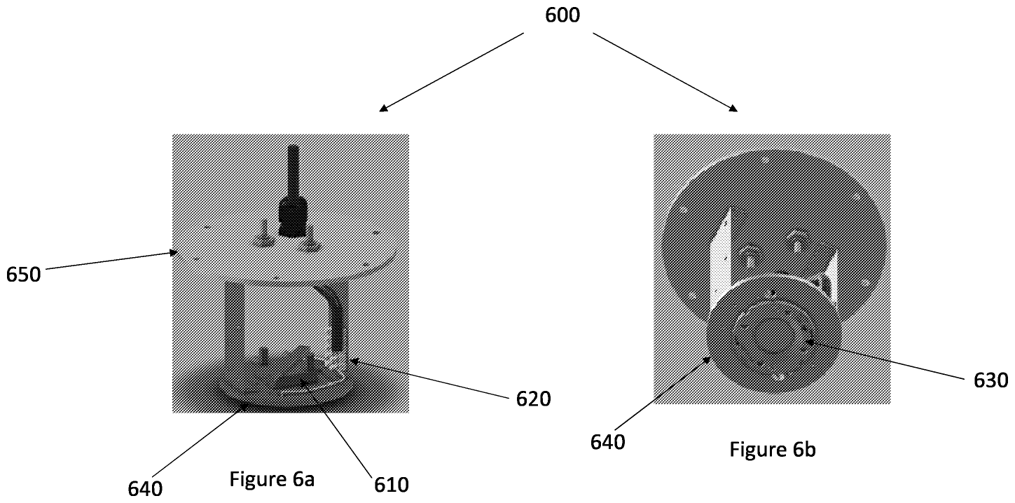

In any of the embodiments described herein, the illumination system may comprise a heat management system located adjacent to the light source, such that it may be within the same housing 104 as the light source. Although the light source 101 may be located outside of the medium, there may still be heat produced and this can affect the growth of algae. To cool the light source 101 a heat management system 102 may be used. It will be appreciated that the heat management system 102 is able to be any system which provides the result of reducing the heat from the light source 101. Examples of such a system 102 include, but are not restricted to, any combination of one or more of the following systems: heat sink, fan, liquid cooling system, heat pipe, heat fin, and air cooled heat exchanger. The choice of heat management system 102 depends on the light source 101 in use and the system outside of the illumination system. For example a liquid cooled heat management system, as illustrated in FIGS. 6a and 6b, may be preferable in a situation where the heated water may be used in a system such as a radiator. It will also be appreciated that the heat management system 102 may just be air if the light source 101 is far enough above the medium.

The light assembly 600 in FIGS. 6a and 6b illustrates one example of the use of a heat management system which could be used any embodiments of the illumination system. The assembly 600 comprises a cooling element 610 through which liquid is able to flow. The cooling element 610 is mounted on a thermally conductive plate 640, in this example a metal plate, on whose other side can be mounted a light source 630. In this example this has the result that the light source 630 is mounted below the thermally conductive plate 640. Consequently, heat from the light source 630 is transferred via the thermal plate 640 to the liquid, thus cooling the light source 630 and also allowing the heated liquid to be utilised in another system if required, for example in a radiator. The light assembly 600 also comprises an electrical system 620 through which electricity is able to be provided to the light source 630.

In the arrangement in which the light assembly 600 is located within the housing 104, the diameter of the plate 640 on which the cooling element is mounted is such that it is able to be inserted inside the housing 104, as illustrated in FIGS. 1c and 1d. The end plate 650 which supports the electrical system, cooling element and light source has a diameter such that it forms the end of the housing 104 and creates a water-tight seal with the housing 104. It will be appreciated that there may be an additional material located on the end plate 650 to form the seal with the housing 104. Although not included in the figure, it will be appreciated that there may be an optical element 103a and/or 103b located on the light assembly 600, or this may be separate. In the case of the light assembly 600 located inside the housing 104, the size of the cone 106 is such that the tip is located below the light source 101 and optics 103a-b. This arrangement has the advantage that the entire illumination system 100 is sealed and is able to be used in applications in which the system may get wet, whereas in the embodiment with a heat management system outside the housing 104, it could be damaged by water.

It will be appreciated that the light assembly 110 and the arrangement of the system respective to the housing 104 can be chosen depending on the application of the system, and the embodiments and figures related to in this application are for illustrative purposes only.

This first embodiment in system 100 may also include optics 103. Such optics are used to achieve the correct light spread and light divergence for the design of the light tube. As illustrated in FIG. 1, the optics 103 may be located below the light source 101. The optics used are chosen with respect to the light source 101. It is preferable that the light entering the housing is collimated to a suitable angle, for example to a divergence half angle of 9-20 degrees. It will be appreciated that the angle of spread for a light source may be chosen with respect to the length of the illuminated housing, for example 20 degrees may be used for a housing of 2 metres, and an angle of 15 degrees may be chosen for a housing of length of 3 metres. It will be appreciated that any optical element 103 which achieves the desired amount of collimation respective to the light source 101 can be used. For the use of an LED it may be preferable to use a collimating optical element 103a, examples of such optical elements include a lens or a reflective parabolic mirror. For the embodiment in which the light source 101 used comprises a laser, a diverging optical element 103b may be used instead, if the light is too collimated. An example of such an element is a diverging lens. The use of optics ensure that the spread of light from the diffusive element 106 in the housing 104 is optimised, and the efficiency is also maximised by minimal loss of light before diffusion.

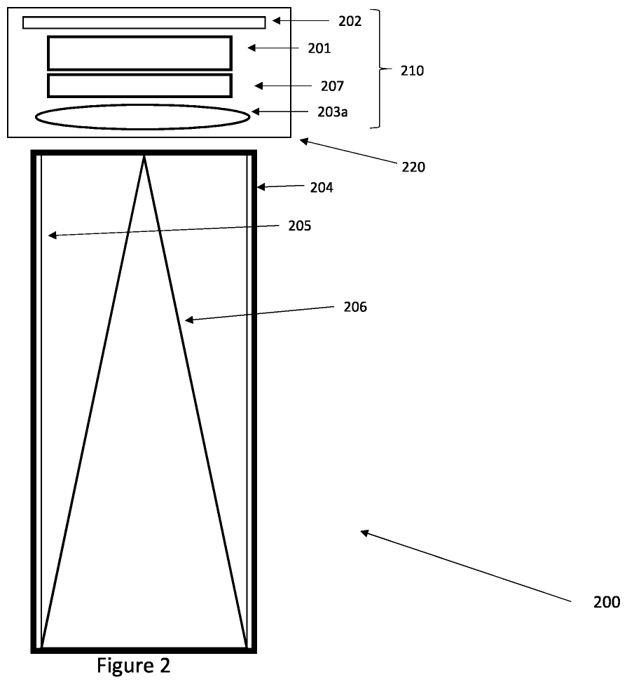

A second embodiment, illustrated in FIG. 2, may contain a variation of the features mentioned in the first embodiment. The apparatus of system 200 contains some of the same features as in 100, and when the features are the same, they have corresponding numbers, varied by the first number being 2 rather than 1. This embodiment comprises a light source 201, close phosphor film 207, heat management system 202, collimating optics 203, housing 204, prismatic film 205 and diffuser 206. Features 202-206, 210 and 220 are the same as in the previous embodiment. The light source 201 in this embodiment is similar to that in the first, with the change that a close phosphor film 207 is used through which the LEDs of the light source 201 are directed. The LEDs in this embodiment are chosen with only a short wavelength emission, for example blue from 410 nm to 460 nm. When the light has passed through the phosphor it will have the final desired spectrum. The desired spectrum can be achieved by the use of the correct type and amount of phosphor, for example it may be a ‘horticultural’ mix of wavelengths of light. The phosphor film 207 is coated directly on the LED light source 201 and is a high efficiency phosphor. Phosphor has the ability to change the wavelength of light, and so for example it can absorb and then re-emit blue wavelengths to produce a red light. This allows an efficient blue LED to be used in this embodiment and by converting the right amount of light it can ensure that a high efficiency is achieved. The phosphor is chosen such that it can withstand the heat of the light source 201 as it is placed directly onto the source, and therefore before the heat management system. This embodiment has the advantage that a more efficient LED can be chosen and the spectrum can be varied by a different type or amount of phosphorous, rather than changing the light source 201 elements.

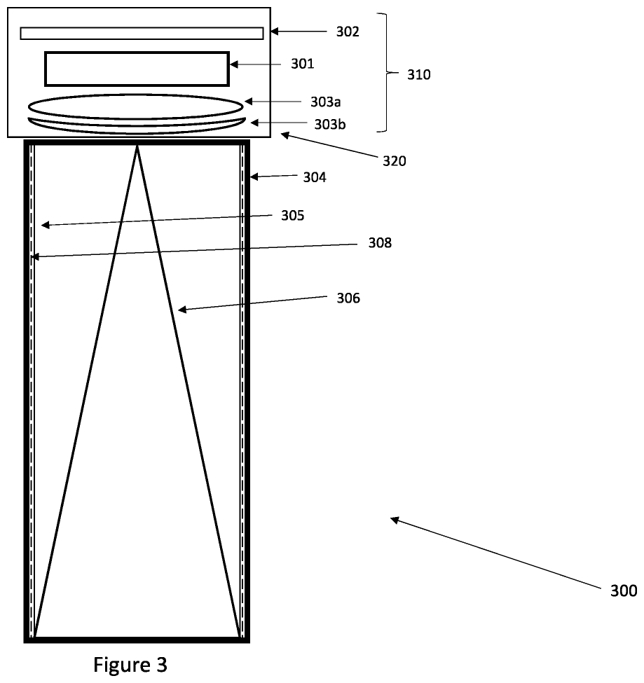

A third embodiment is a further variation of the first two embodiments. This is illustrated in FIG. 3 in which system 300 has been illustrated. As before, the features with corresponding numbers are the same and have the same effects as the previous two. The embodiment comprises a heat management system 302, a light source 301, a remote phosphor film 308, a diffuser 306, a housing 304, optics 303a-b and a prismatic film 305. This embodiment differs in its phosphor feature and its light source 301. The light source 301 is made of one or more blue (420-480 nm) laser diodes or LEDs. The light is converted to the desired spectrum by use of a remote phosphor film 308 located on the housing. The phosphor film 308 consists of a substrate and a coated material. The material consists of a UV curable acrylate in which inorganic phosphor has been suspended. The inorganic phosphor combination is uniformly distributed either in a clear resin which has been coated on a clear substrate, for example PET, PMMA or PC, and cured for example using UV cure or the phosphor is uniformly distributed in the material of the film itself. The phosphor converts between blue light (420-480 nm) and red light (600-680 nm). The layer is preferably located on the inside of the housing 304 as a film, although it will be appreciated that a phosphor layer could be located on the diffuser 306 in a coating. The phosphor film 308 is optimally located outside of the prismatic film 305 (if included), such that it is located between the prismatic film 305 and the housing 304. This location is optimal as the phosphor film 308 can scatter the light and destroy the uniformity by reflecting the light before it reaches the prismatic film 305, and so it is preferable that the light reaches the prismatic film 305 first. The phosphor film 308 is arranged such that it fully covers the inner surface of the housing 304. The advantage of using a remote phosphor film 308 is that the film does not have to be able to withstand the heat that the close phosphor 307 of the previous embodiment does. This allows a cheaper or organic phosphor to be used.

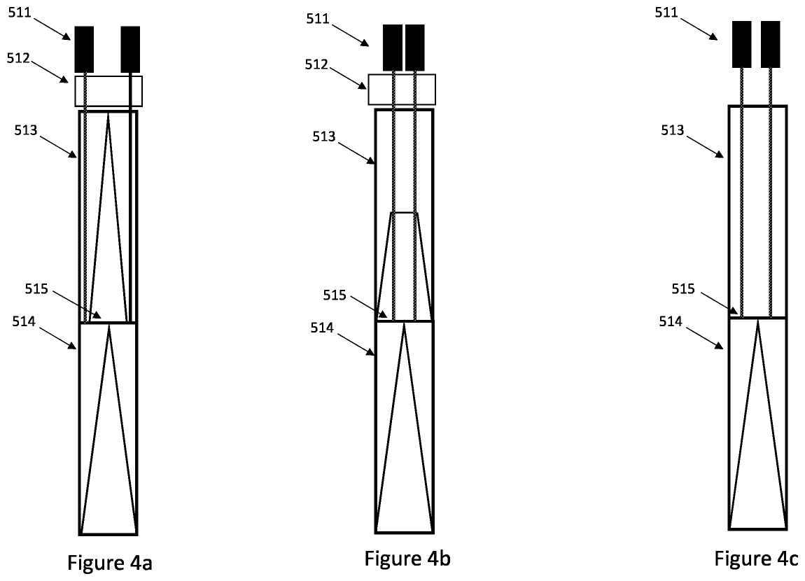

In a fourth embodiment shown in FIGS. 4a-c, the system comprises the same features as in the previous embodiment, such as a heat management system, a light source 511, optics and a housing 514 comprising a remote phosphor film, a diffuser and a prismatic film. However, in this embodiment the light source 511 is preferably a single or an array of solid state lasers such as laser diodes which are arranged to emit along the axis of the light tube. The emission wavelength is chosen to enable the generation of a suitable wavelength spectrum when used in conjunction with the phosphor coated film. An example of a suitable light source 511 may be a high power blue laser diode emitting between 410 and 450 nm. The laser sources 501 may be single or multiple and may be directed along the centre of the housing, FIG. 5b, or in a ring, FIG. 5a, within the outer parts of the housing. This light source 511 is highly collimated already, preferably with a spread less than one degree, and therefore no collimating optics are required and instead the system may comprise diverging optics. A diverging optical plate may be located immediately after the light source or may be located at some distance down the light tube and diverges the laser light to the correct divergence angle for the housing. For example, this angle may be a half maximum angle of 9-15 degrees.

Further variations of the fourth embodiment utilise the apparatus in the previous embodiments, whilst also including an additional housing 513, as is illustrated in FIGS. 4a-c. The first example of the fourth embodiment, illustrated in FIG. 4a, comprises an additional housing 513 placed between the housing 514 illuminated by the laser light source and the laser 511. In this arrangement the additional housing 513 on the top can be illuminated by any of the previous embodiments, in which the housing is also described by any of the previous embodiments. The upper section 513 diverges light into the medium as previously described. The laser 511, which is part of the system described in the fourth embodiment, is located above the additional light source 512. When arranged in the ring formation, as illustrated in FIG. 5a, the light passes through the upper housing 513 without interacting with its optical components until it reaches a diverging optical place 515 mounted between the upper housing 513 and the light illuminating housing 514 designed by the fourth embodiment. The diffuser of the upper housing 513 may not completely fill the base of the housing 513 such that there is a ring through which the laser light can pass through to reach the diverging optics 515 between the housings. Here, the laser light diverges to the required angle such that it will interact with the diffuser and illuminate the lower part of the algae medium. It will be appreciated that the optical elements may be positioned anywhere in the system such that they give the desired divergence, and they are not limited to being placed in between the housings.

Another possible variation of the above embodiment is the case in which the one or more lasers are arranged in a circular channel, FIG. 5b, to illuminate the centre of the housing, illustrated in FIG. 4b. In this case the diffuser in the above housing 513 does not taper to a point and instead tapers to the diameter of the central channel of the light source, forming a frustum shape. The light from the laser source 511 therefore passes through the centre of the cone and pass to their diverging optics 515 at the base of the channel.

An alternative variation of the fourth embodiment is illustrated in FIG. 4c, in which the additional housing 513 is empty and air filled. The laser light can be arranged in any way to illuminate the lower lighting tube 514. It is also possible that the above housing 513 also comprises an internally reflecting film mounted just within the cylinder. In this embodiment all of the illumination takes place in the lower section 514, thereby enabling illumination of a surrounding volume some way below the surface of the liquid volume.

In the figures above, the system is illustrated in which it extends longitudinally. However, it will be appreciated by a person skilled in the art that the system need not be arranged vertically and the system could be orientated laterally such that the light source and housing are still in the same arrangement respective to each other, and the same result would be obtained.

As will be appreciated by someone skilled in the art, although the above embodiments have been described in terms of an algae growing medium, it is able to be used to illuminate any medium.

1. An illumination system for a photosynthetic organism-growing medium comprising:
a waterproof housing arranged to be inserted into photosynthetic organism-growing medium and having a length along a longitudinal axis that is greater than a width;
a light source arranged to provide illumination along the length of the housing; and
a diffuser arranged within the waterproof housing, the diffuser having a narrow end directed towards the light source and a wider end away from the light source and having a diffusive reflective surface arranged to diffusively reflect light from the light source to outside the housing.

2.-60. (canceled)

61. An illumination system according to claim 1, wherein the diffuser is a cone.

62. An illumination system according to claim 1, wherein the diffusive reflective surface of the diffuser comprises a diffusively reflective paint, wherein optionally the paint contains a high concentration of barium sulphate.

63. An illumination system according to claim 1, wherein the housing comprises a light redirecting film.

64. An illumination system according to claim 63, wherein the light redirecting film is located around the inside perimeter of the housing.

65. An illumination system according to claim 63, wherein the light redirecting film is a prismatic film with specified prism angles.

66. An illumination system according to claim 65, wherein the prisms run along the length of the film in the axial direction of the housing.

67. An illumination system according to claim 66, wherein the prisms face inwards.

68. An illumination system according to claim 1, wherein the system comprises a heat management system.

69. An illumination system according to claim 68, wherein the heat management system is located above the medium.

70. An illumination system according to claim 68, wherein the heat management system comprises one or more of a heat sink, a fan, a liquid cooling system, a heat pipe, a heat fin, and an air cooled heat exchanger.

71. An illumination system according to claim 1, wherein the light source is arranged outside the housing and arrange to direct light into the housing.

72. An illumination system according to claim 1, wherein the light source is a laser or an LED.

73. An illumination system according to claim 1, wherein the light source emits a mix of red and blue light.

74. An illumination system according to claim 1, wherein the system comprises a phosphor layer, wherein optionally, the phosphor produces a desired light spectrum and wherein optionally the desired spectrum is a mix of blue and red light.

75. An illumination system according to claim 74, wherein the light source is an LED, and the phosphor layer is mounted directly on the light source.

76. An illumination system according to claim 74, wherein the phosphor layer is a film on the housing, wherein optionally the phosphor film is located adjacent to the inside of the housing.

77. An illumination system according to claim 74, wherein the phosphor film consists of a substrate and a coated material.

78. An illumination system according to claim 77, wherein the material consists of a UV curable acylate in which inorganic phosphor has been suspended.

79. An illumination system according to claim 1, wherein there is an additional housing located between the light source and housing, wherein optionally the additional housing contains an internally reflecting film mounted within the housing.
